SEQUENCE LISTING (4 sequences)

SEQ ID NO: 1 - DNA, 53 bp, synthetic construct
  source 1..53: mol_type = other DNA
cgacggtggcctattaaatagctttagtttaagaaaagtaatagggggtgtcg

SEQ ID NO: 2 - DNA, 15 bp, synthetic construct
  source 1..15: mol_type = other DNA
aataggccaccgtcg

SEQ ID NO: 3 - DNA, 19 bp, synthetic construct
  source 1..19: mol_type = other DNA
tcttaaactaaagctattt

SEQ ID NO: 4 - DNA, 19 bp, synthetic construct
  source 1..19: mol_type = other DNA
cgacaccccctattacttt

(19) United States
(12) Patent Application Publication
(10) Pub. No.: US 2026/0243782 A1
(43) Pub. Date: Aug. 20, 2026

(54) KIT AND METHOD OF DETERMINING THE PRESENCE OR AMOUNT OF CREATININE

(71) Applicant: Hong Kong Centre for Cerebro-Cardiovascular Health Engineering Limited, Pak Shek Kok (HK)

(72) Inventors: Chenyu Cui, Pak Shek Kok (HK); Ting-hsuan Chen, Pak Shek Kok (HK)

(21) Appl. No.: 19/055,017

(22) Filed: Feb. 17, 2025

(51) Int. Cl. G01N 33/70; G01N 33/53

(52) U.S. Cl. CPC G01N 33/70; G01N 33/5308

(57) ABSTRACT

A kit for determining the presence and/or amount of creatinine in a sample including: (i) an aptamer having a binding activity against creatinine; and (ii) a blocker sequence which is complementary to a portion of the aptamer for forming an aptamer-blocker duplex, wherein the blocker sequence is released from the aptamer when the aptamer binds to the creatinine in the sample. The invention also relates to a method of determining the presence and/or amount of creatinine in a sample including a step of contacting at least a portion of the sample with the aptamer-blocker duplex.

### SEQUENCE LISTING

The Sequence Listing file entitled “sequencelisting” having a size of 4277 bytes and a creation date of Feb. 14, 2025, that was filed with the patent application is incorporated herein by reference in its entirety.

### TECHNICAL FIELD

The present invention relates to an assay of an analyte of interest, and particularly, although not exclusively, to an assay for detection of creatinine in a sample.

### BACKGROUND

Creatinine is a waste product from muscle metabolism, and is mainly excreted by the kidneys. Its levels in blood and urine are critical indicators of kidney function. For example, the elevated levels of creatinine in blood can indicate impaired kidney function or kidney disease. For individuals with known kidney disease or those at risk, regular monitoring of creatinine levels help track the progression of the disease and the effectiveness of treatments.

Traditional methods for detecting creatinine, such as Jaffe reaction, enzyme-based assays, and high-performance liquid chromatography (HPLC), often involve complex procedures, lengthy analysis times, and expensive equipment. For example, the enzyme-based assays require specific conditions to maintain enzyme activity and the reagents used to perform the assays are expensive. Jaffe reaction is less specific and could be affected by other substance in the sample, resulting in false positives or artificially elevated readings.

Accordingly, there remains a need for an improved method for determining creatinine a sample with a relatively high specificity, and is a more cost effective.

### SUMMARY OF THE INVENTION

In accordance with a first aspect of the present invention, there is provided a kit for determining the presence and/or amount of creatinine in a sample, comprising:
(i) an aptamer having a binding activity against creatinine; and
(ii) a blocker sequence which is complementary to a portion of the aptamer for forming an aptamer-blocker duplex, wherein the blocker sequence is released from the aptamer when the aptamer binds to the creatinine in the sample.

In an embodiment of the first aspect, the aptamer is a DNA aptamer.

In an embodiment of the first aspect, the aptamer comprises a sequence of SEQ ID NO: 1 or a sequence having at least 80% sequence identity to SEQ ID NO: 1, i.e. CGACGGTGGCCTATTAAATAGCTTTAGTTTAAGA AAAGTAATAGGGGGTGTCG.

In an embodiment of the first aspect, the blocker sequence is between about 10-25 nucleotides in length.

In an embodiment of the first aspect, wherein the blocker sequence is between about 15-20 nucleotides in length.

In an embodiment of the first aspect, the blocker sequence is complementary to about 25%-40% of the aptamer.

In an embodiment of the first aspect, the blocker sequence comprises a sequence selected from SEQ ID NO. 2, SEQ ID NO. 3, SEQ ID NO. 4, or a sequence having at least 80% sequence identity to any of SEQ ID NO. 2 to SEQ ID NO. 4. In a preferred embodiment, the blocker sequence comprises a sequence of SEQ ID NO. 4.

In an embodiment of the first aspect, the kit further comprises an assay panel immobilized with a probe molecule for capturing the blocker sequence in free form.

In accordance with a second aspect of the invention, there is provided a method of determining the presence and/or amount of creatinine in a sample, comprising:
(i) contacting at least a portion of the sample with an aptamer-blocker duplex, wherein the aptamer-blocker duplex is formed by an aptamer having a binding activity against creatinine, and a blocker sequence which is complementary to a portion of the aptamer;
(ii) allowing the aptamer-blocker duplex to react with creatinine present in the sample under conditions for releasing the blocker sequence; and
(iii) determining the presence or amount of the blocker sequence in free form in the resultant mixture, or determining the amount of aptamer-blocker duplex in the resultant mixture.

In an embodiment of the second aspect, the aptamer is a DNA aptamer.

In an embodiment of the second aspect, the aptamer comprises a sequence of SEQ ID NO: 1 or a sequence having at least 80% sequence identity to SEQ ID NO: 1.

In an embodiment of the second aspect, the blocker sequence is between about 10-25 nucleotides in length. Preferably, the blocker sequence is between about 15-20 nucleotides in length.

In an embodiment of the second aspect, the blocker sequence is complementary to about 25%-40% of the aptamer.

In an embodiment of the second aspect, the blocker sequence comprises a sequence selected from SEQ ID NO. 2, SEQ ID NO. 3, SEQ ID NO. 4, or a sequence having at least 80% sequence identity to any of SEQ ID NO. 2 to SEQ ID NO. 4. Preferably, the blocker sequence comprises a sequence of SEQ ID NO. 4.

In an embodiment of the second aspect, the sample is applied on an assay panel comprising a surface immobilized with a probe molecule for capturing the blocker sequence in free form.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described, by way of example, with reference to the accompanying drawings in which:

FIG. 1 is a schematic diagram showing the reaction of an aptamer-blocker duplex in accordance with one embodiment of the present invention, with or without creatinine in a sample;

FIG. 2 is a schematic diagram of the structure of the aptamer in accordance with one embodiment of the present invention;

FIG. 3A is a schematic diagram of the structure of an aptamer-blocker duplex in accordance with one embodiment of the present invention, in which blocker sequence 1 (SEQ ID NO. 2) is used to form the duplex;

FIG. 3B is a schematic diagram of the structure of an aptamer-blocker duplex in accordance with another embodiment of the present invention, in which blocker sequence 2 (SEQ ID NO. 3) is used to form the duplex;

FIG. 3C is a schematic diagram of the structure of an aptamer-blocker duplex in accordance with a further embodiment of the present invention, in which blocker sequence 3 (SEQ ID NO. 4) is used to form the duplex;

FIG. 4 illustrates a photo of the results obtained from agarose gel electrophoresis with different resulted mixtures, i.e. an aptamer only (Lane 1), a blocker sequence only (Lane 2) an aptamer-blocker duplex (Lane 3), and an aptamer-blocker duplex with creatinine (Lane 4).

### DETAILED DESCRIPTION OF THE PREFERRED EMBODIMENTS

Unless otherwise defined, all technical terms used herein have the same meaning as commonly understood by one skilled in the art to which the invention belongs.

As used herein, “comprising” means including the following elements but not excluding others. “Essentially consisting of” means that the material consists of the respective element along with usually and unavoidable impurities such as side products and components usually resulting from the respective preparation or method for obtaining the material such as traces of further components or solvents. “Consisting of” means that the material solely consists of, i.e. is formed by the respective element. As used herein, the forms “a”, “an”, and “the”, are intended to include the singular and plural forms unless the context clearly indicates otherwise.

The present invention in an aspect provides a kit for determining the presence and/or amount of creatinine in a sample. The kit comprises
(i) an aptamer having a binding activity against creatinine; and
(ii) a blocker sequence which is complementary to a portion of the aptamer for forming an aptamer-blocker duplex.
   Preferably, when the aptamer-blocker duplex interacts with creatinine in the sample, i.e. when the aptamer binds to the creatinine, the structure of the aptamer-blocker duplex changes and releases the blocker sequence.

The term “aptamer” as used herein refers to a relatively short, single-stranded nucleic acid that can fold into a tertiary structure for specifically binding to a target analyte. In this invention, the aptamer is provided to target creatinine so as to determine the presence or amount of it in a sample, thereby assisting the assessment of renal function of a subject.

The aptamer of the present invention is preferably a DNA aptamer. In an embodiment, the aptamer has a sequence of between about 40-70 nucleotides in length, or between about 50-60 nucleotides in length, or particularly 53 nucleotides in length. In a preferred embodiment, the aptamer comprises a sequence of SEQ ID NO. 1 or a sequence having at least 80%, 85%, 90%, 95%, or 98% sequence identity to SEQ ID NO. 1. It would be appreciated that modification could be made to SEQ ID NO. 1 without suppressing the binding activity of the aptamer against creatinine.

The blocker sequence of the present invention refers to a sequence that is complementary to at least a portion of the aptamer and it is able to form a duplex with the aptamer via hybridization. In an embodiment, the blocker sequence is shorter than the aptamer in length. In particular, the blocker sequence may be between about 10-25 nucleotides in length, between about 15-20 nucleotides in length, or about 19 nucleotides in length. In an embodiment, the blocker sequence is complementary to about 25%-40% of the aptamer. Accordingly, the aptamer-blocker duplex can interact with creatinine at the portion where the blocker sequence is not bound to the aptamer, and trigger the release of the blocker sequence in free form for subsequent determination.

In an embodiment, the blocker sequence of the present invention comprises a sequence selected from SEQ ID NO. 2, SEQ ID NO. 3, SEQ ID NO. 4, or a sequence having at least 80%, 85%, 90%, 95% sequence identity to any of SEQ ID NO. 2 to SEQ ID NO. 4. In a preferred embodiment, the blocker sequence comprises or consists of a sequence of SEQ ID NO. 4.

The sample as used in the present invention may be a biological fluid obtained from a subject, preferably a mammal such as a human, or may be obtained from cells or a cell population. In an embodiment, the sample may be a biological fluid selected from the group consisting of serum, blood, urine, transcellular fluid, interstitial fluid, saliva, exudates, fluid collected through microdialysis and other fluids from mammal susceptible of containing histamine. Preferably, the sample is serum, urine, transcellular fluid or fluid collected through microdialysis. In a particular embodiment, the sample is serum, or urine.

In another aspect, the present invention also provides a method of determining the presence and/or amount of creatinine in a sample. The method includes the following steps:
(i) contacting at least a portion of the sample with the aptamer-blocker duplex as described above;
(ii) allowing the aptamer-blocker duplex to react with creatinine present in the sample under conditions for releasing the blocker sequence; and
(iii) determining the presence or amount of the blocker sequence in free form in the resultant mixture, or determining the amount of aptamer-blocker duplex in the resultant mixture.
   Before step (i), the aptamer-blocker duplex may be prepared by the following steps:
(a) dissolving the aptamer as described above in a nuclease-free solution, e.g. nuclease-free water;
(b) subjecting the solution of (a) to a heating protocol followed by a cooling procedure, so as to allow the aptamer to form a 3D structure;
(c) mixing the blocker sequence as described above to the obtained aptamer, and subjecting the mixture under hybridization condition, thereby forming the aptamer-blocker duplex.

In an embodiment, the step (b) includes heating the aptamer at 90° C. to 95° C. for about 1-10 minutes or about 3-5 minutes, followed by a gradual cooling to 25° C. over 10-20 minutes. Preferably, the step (b) includes heating at 95° C., followed by a gradual cooling to 25° C. at a rate of 0.5° C./min.

In step (c), to facilitate the hybridization, the aptamer and the blocker sequence are mixed in a buffer solution, and the mixture is placed at room temperature for about 15 minutes to about 1 hour, about 15 minutes to about 30 minutes, or about 30 minutes to allow sufficient time for hybridization. In a specific embodiment, the buffer solution is HEPES buffer having 20 mM HEPES, 1M sodium chloride (NaCl), 10 mM magnesium chloride (MgCl₂), 5 mM potassium chloride (KCl), and has a pH of 7.5. It would be appreciated that other buffer solutions suitable for hybridization could be applied. The present method is effective without the need for highly stringent conditions for preparing the materials or reagents for detection.

As described above, when the aptamer-blocker duplex interacts with creatinine present in the sample, the aptamer portion will bind to the creatinine, and such a binding trigger structural change of the duplex. The blocker sequence is then released from the duplex, and present in free form in the reaction mixture. By determining the presence or amount of the blocker sequence in the reaction mixture, the presence and/or amount of creatinine can then be determined. The present method is advantageous due to its simpler procedures and greater cost-effectiveness.

Alternatively, it is also feasible to determine the amount of aptamer-blocker duplex in the reaction mixture so as to determine the creatinine. For example, the determination could be done via running agarose gel electrophoresis. Other suitable methods could also be applied in the present invention.

In a further embodiment, the method may involve use of an assay panel such as a microfluidic channel. The panel may be provided with a surface coated or immobilized with one or more probe molecules for capturing the blocker sequence in free form. Once the probe captures the free blocker sequence, it will give detectable signal for measurement such as electrochemical signal or fluorescence.

It would be appreciated that the kit according to the present invention may also include an assay panel as described above for rapid measurement.

The invention further relates to a method of determining the health condition of a kidney of a subject by using the above kit or method.

### EXAMPLES

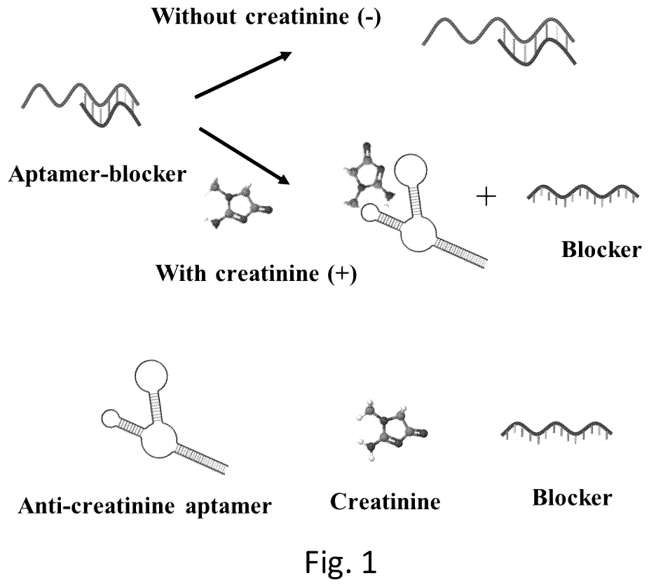

FIG. 1 shows the concept of using an aptamer-blocker duple of the present invention for detection of creatinine in a sample. As illustrated, when there is creatinine in the sample, the duplex will bind to the creatinine molecule and release the blocker sequence. When creatinine is absent, the structure of the duplex remains unchanged.

### Example 1

### Synthesis of Aptamer and Blocker Sequences

The inventors prepared the aptamer and blocker sequences according to Table 1.

TABLE 1
Anti-creatinine aptamer and blocker sequences

| Strand Name | Sequence (5′ to 3′) |
|---|---|
| Aptamer | CGACGGTGGCCTATTAAAT |
| | AGCTTTAGTTTAAGAAAAG |
| | TAATAGGGGGTGTCG |
| | (SEQ ID NO. 1) |
| Blocker sequence 1 | AAT AGG CCA CCG TCG |
| | (SEQ ID NO. 2) |
| Blocker sequence 2 | TCT TAA ACT AAA GCT |
| | ATT T |
| | (SEQ ID NO. 3) |
| Blocker sequence 3 | CGA CAC CCC CTA TTA |
| | CTT T |
| | (SEQ ID NO. 4) |

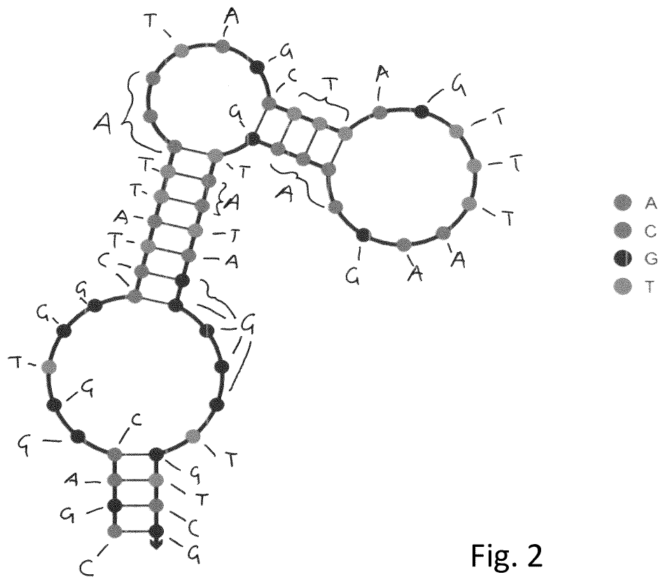

FIG. 2 illustrated the structure of the aptamer, based on the calculation using the NUPACK (https://www.nupack.org/). The aptamer oligonucleotide was synthesized by Sangon (Shanghai, China) and subsequently dissolved in nuclease-free water. It was then subjected to a heating protocol at 95° C., followed by a gradual cooling to 25° C. at a rate of 0.5° C./min to ensure the formation of the desired three-dimensional structure.

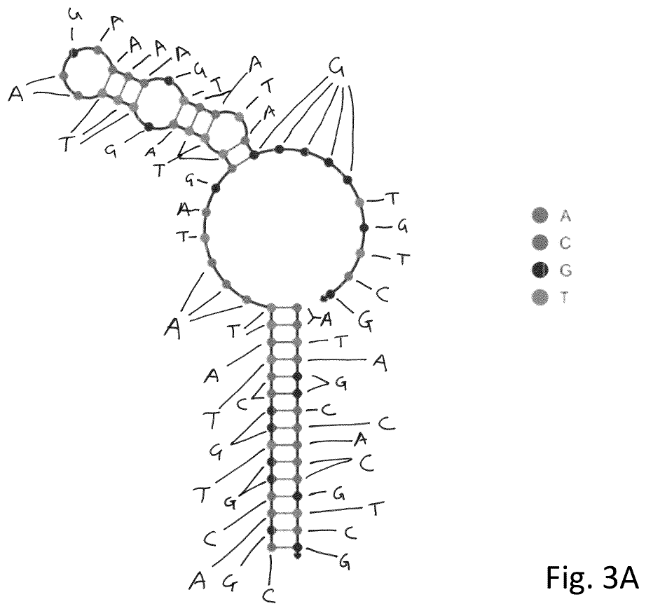

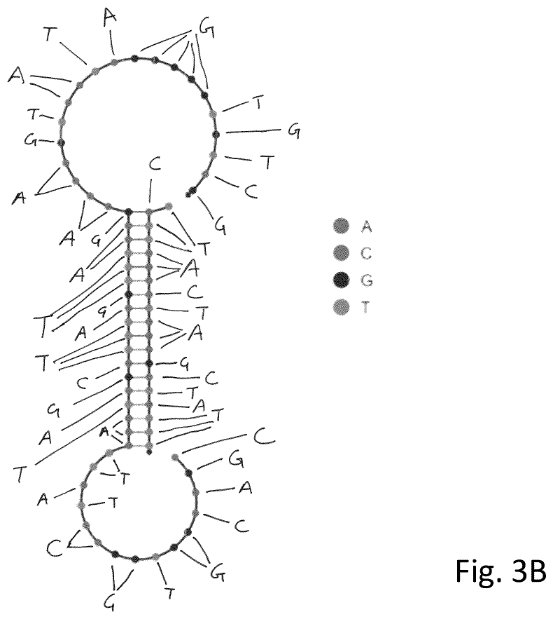

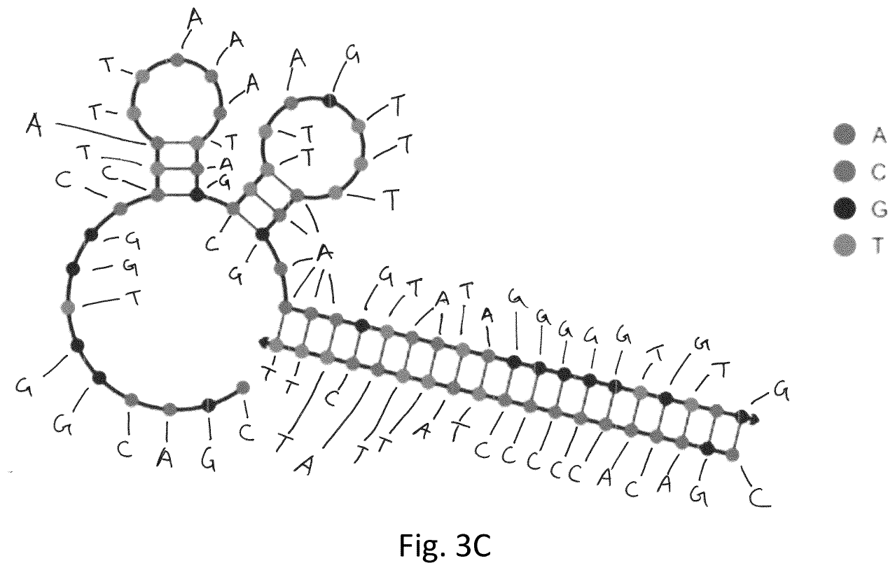

The inventors designed three blocker sequences for hybridizing with different regions of the aptamer. FIG. 3A-3C shows the aptamer-blocker hybridization simulation structures.

### Example 2

### Validation Using Agarose Gel Electrophoresis

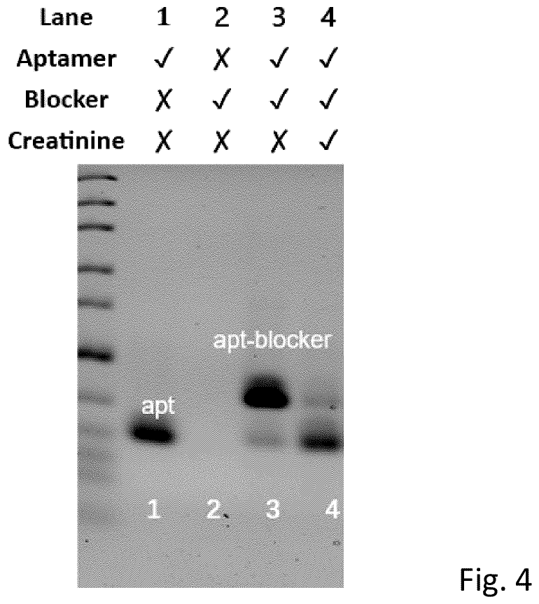

The inventors found that the blocker sequence 3 achieves the best releasing performance. The results of FIG. 4 show that the blocker sequence 3 (i.e. SEQ ID NO. 4) hybridizes effectively with the aptamer and exhibits a high release efficiency.

As shown in FIG. 4, in Lane 1, the band indicates the presence of the aptamer. Lane 2 shows no visible band due to the short length of the blocker sequence. The aptamer-blocker duplex resulted in a more pronounced band in Lane 3, demonstrating successful hybridization. Upon the introduction of creatinine, the band corresponding to the aptamer-blocker complex became lighter, while the band for the aptamer became more prominent. This observation confirms the successful release of the blocker strand and validates the feasibility of the creatinine detection scheme.

Based on the above, the inventors developed a more cost-effective approach to determine the present or level of creatinine. The present invention utilizes a blocker sequence that hybridizes with an aptamer against creatinine. When creatinine binds to the aptamer, it releases the blocker sequence. It is believed that the released blocker sequence can be seamlessly integrated into a wide range of sensing platforms, including electrochemical sensors, optical devices, and point-of-care systems, significantly broadening their applications in both diagnostics and research.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A kit for determining the presence and/or amount of creatinine in a sample, comprising:
(i) an aptamer having a binding activity against creatinine; and
(ii) a blocker sequence which is complementary to a portion of the aptamer for forming an aptamer-blocker duplex, wherein the blocker sequence is released from the aptamer when the aptamer binds to the creatinine in the sample.

2. The kit of claim 1, wherein the aptamer is a DNA aptamer.

3. The kit of claim 1, wherein the aptamer comprises a sequence of SEQ ID NO: 1 or a sequence having at least 80% sequence identity to SEQ ID NO: 1.

4. The kit of claim 1, wherein the blocker sequence is between about 10-25 nucleotides in length.

5. The kit of claim 4, wherein the blocker sequence is between about 15-20 nucleotides in length.

6. The kit of claim 1, wherein the blocker sequence is complementary to about 25%-40% of the aptamer.

7. The kit of claim 1, wherein the blocker sequence comprises a sequence selected from SEQ ID NO. 2, SEQ ID NO. 3, SEQ ID NO. 4, or a sequence having at least 80% sequence identity to any of SEQ ID NO. 2 to SEQ ID NO. 4.

8. The kit of claim 7, wherein the blocker sequence comprises a sequence of SEQ ID NO. 4.

9. The kit of claim 1, further comprising an assay panel immobilized with a probe molecule for capturing the blocker sequence in free form.

10. A method of determining the presence and/or amount of creatinine in a sample, comprising:
(i) contacting at least a portion of the sample with an aptamer-blocker duplex, wherein the aptamer-blocker duplex is formed by an aptamer having a binding activity against creatinine, and a blocker sequence which is complementary to a portion of the aptamer;
(ii) allowing the aptamer-blocker duplex to react with creatinine present in the sample under conditions for releasing the blocker sequence; and
(iii) determining the presence or amount of the blocker sequence in free form in the resultant mixture, or determining the amount of aptamer-blocker duplex in the resultant mixture.

11. The method of claim 10, wherein the aptamer is a DNA aptamer.

12. The method of claim 10, wherein the aptamer comprises a sequence of SEQ ID NO: 1 or a sequence having at least 80% sequence identity to SEQ ID NO: 1.

13. The method of claim 10, wherein the blocker sequence is between about 10-25 nucleotides in length.

14. The method of claim 13, wherein the blocker sequence is between about 15-20 nucleotides in length.

15. The method of claim 10, wherein the blocker sequence is complementary to about 25%-40% of the aptamer.

16. The method of claim 10, wherein the blocker sequence comprises a sequence selected from SEQ ID NO. 2, SEQ ID NO. 3, SEQ ID NO. 4, or a sequence having at least 80% sequence identity to any of SEQ ID NO. 2 to SEQ ID NO. 4.

17. The method of claim 16, wherein the blocker sequence comprises a sequence of SEQ ID NO. 4.

18. The method of claim 10, wherein the sample is applied on an assay panel comprising a surface immobilized with a probe molecule for capturing the blocker sequence in free form.
